(19) United States
(12) Patent Application Publication
(10) Pub. No.: US 2005/0256168 A1
(43) Pub. Date: Nov. 17, 2005

(54) Compositions for oral administration for the treatment of interferon-responsive disorders

(76) Inventors: Timothy M. Block, Doylestown, PA (US); Anand Mehta, Lansdale, PA (US); Xuanyong Lu, Horsham, PA (US); Raymond A. Dwek, Oxford (GB)

Correspondence Address: FOX ROTHSCHILD O'BRIEN & FRANKEL LLP;PRINCETON PIKE CORPORATE CENTER, 997 LENOX DRIVE, BUILDING 3, LAWRENCEVILLE, NJ, 08648, US

(21) Appl. No.: 11/116,088

(22) Filed: Apr. 27, 2005

Related U.S. Application Data
(60) Provisional application No. 60/566,380, filed on Apr. 28, 2004.
(60) Provisional application No. 60/607,765, filed on Sep. 7, 2004.

(51) Int. Cl. A61K 31/445; A61K 31/4015

(52) U.S. Cl. 514/327; 514/425

(57) ABSTRACT

Compositions and methods of treatment of interferon-responsive disorders are provided wherein the methods, for example, comprise orally administering a composition which comprises a therapeutically effective amount of at least one alkylated imino sugar having an alkylated side chain, and a pharmaceutically acceptable carrier.

This application derives priority from U.S. provisional application 60/566,380, filed on Apr. 28, 2004 and U.S. provisional application 60/607,765, filed Sep. 7, 2004.

### FIELD OF THE INVENTION

The present invention relates to compositions for oral administration. Particularly, methods of treatment of an interferon-responsive disorder comprising orally administering a composition which comprises a therapeutically effective amount of at least one alkylated imino sugar having an alkylated side chain, and a pharmaceutically acceptable carrier, wherein symptoms of an interferon-responsive disorder are controlled.

### BACKGROUND OF THE INVENTION

The human lymphatic system and other organs are regulated in part by naturally produced, human polypeptides called “cytokines”. Many cytokines are produced by cells of the immune system, in response to infection, and these cytokines are thought to be, in part, an important way in which the human body fights off infection. In response to the cytokines themselves or other elements induced by infection, mammalian cells produce cytokines and other intracellular biochemical agents, collectively recognized as the “innate host” defense response or pathway. It is called “innate” since prior immunization and lymphocyte “memory” are not necessarily involved.

There are many illnesses that have been shown to benefit from treatment with cytokines. In particular, and of great relevance to our invention, described here, is the benefit that results from treatment with protein members of the interferon family. Viral hepatitis B and C, the major etiologies of human cirrhosis and liver cancer, are responsive, and interferon alpha has been approved by the US FDA for treatment of chronic hepatitis B and C.

Interferon beta has been approved by the U.S. FDA for the treatment of Multiple Sclerosis, a quality of life-disturbing, sometimes life-ending progressive neurological disease of great morbidity.

Interferons have been shown to be anti-proliferative for a number of malignancies and have been used beneficially to treat the following human disorders: neuroblastomas, multiple myelomas, malignant melanomas, kidney tumors, carcinoid tumors, and ovarian cancer. Rheumatoid arthritis and severe respiratory syndrome have also been shown to be beneficially treated with interferons (Schreiner B, Mitsdoerffer M, Kieseier B C, Chen L, Hartung H P, Weller M, Wiendl H, Cinatl J Jr, Michaelis M, Scholz M, Doerr H W; Interferon-beta enhances monocyte and dendritic cell expression of B7-H1 (PD-L1), a strong inhibitor of autologous T-cell activation: relevance for the immune modulatory effect in multiple sclerosis; Van Holten J, Reedquist K, Sattonet-Roche P, Smeets T J, Plater-Zyberk C, Vervoordeldonk M J, Tak P P. Treatment with recombinant interferon-beta reduces inflammation and slows cartilage destruction in the collagen-induced arthritis model of rheumatoid arthritis. Arthritis Res Ther. 2004;6(3):R239-R249).

The broad spectrum of syndromes that could benefit from enhancement of dendritic cell activity would also be expected to benefit from interferon therapy. Taken together, interferons have a broad spectrum of therapeutic activity. There are, however, very significant limitations to the use of interferons. To begin with, interferons, as with most, if not all, cytokines, must be given to the patient by parenteral routes of administration (non oral routes). Thus, the patient must be injected or inject themselves by syringe or line or other internal, non oral, mechanisms. This is a very serious limitation, since injections must usually be given multiple times per week, which is inconvenient, sometimes painful, and anxiety-producing.

Another limitation to interferon use relates to the “side effects” or “adverse reactions” that accompany its use. These side affects are often use-limiting, with many people choosing to avoid interferon because of the side affects. The side affects certainly limit the dose of interferon that will or can be taken by a person, and this goes to the issue of efficacy, since doses (amounts) of interferon that would otherwise be efficacious can not or will not be taken, because those amounts cause adverse side effects. The side effects range from fairly trivial (headaches, minor discomforts perhaps associated with injection) to neurological, nightmares, fatigue, ill feeling, nausea, pains, through serious dose limiting fevers, and even psychotic episodes.

Side affects and the need for injection are therefore major limitations to the use of interferons and cytokines.

Interferon alpha and beta achieve their beneficial effects by inducing other cellular biochemical messengers, such as 2-5-O-A-synthetase and the so called “p40” and p69 human gene products (Hovnanian A, R. D., Levy E R, Mattei M G, Hovanessian A G. 1999. The human 2′,5′-oligoadenylate synthetase-like gene (OASL) encoding the interferon-induced 56-kDa protein maps to chromosome 12q24.2 in the proximity of the 2′,5′-OAS locus. Genomics 56:362-373).

What is needed, therefore, is a drug that can induce interferon and other interferon-induced biochemical messengers, that can be taken orally and would eliminate the discomforts and limitations associated with the currently available, injection dependent, interferons. A drug that induces only a subset of the interferon-inducible genes but still has antiviral or anti-proliferative effects, would likely retain efficacy for many disorders, while losing some of the unwanted side effect. The invention described herein addresses these needs.

### SUMMARY OF THE INVENTION

The present invention is directed to methods of treatment of pathophysiological conditions by induction of the innate host defense pathway, of which interferon treatable conditions are a sub-set, comprising orally or parenterally administering a composition which comprises a therapeutically effective amount of at least one alkylated imino sugar having an alkylated side chain, and a pharmaceutically acceptable carrier.

The current invention is further directed to a container comprising a composition which comprises a therapeutically effective amount of at least one alkylated imino sugar having an alkylated side chain and a pharmaceutically acceptable carrier; and, instructions for oral administration of the composition for the treatment of an interferon-responsive disorder.

The invention is further directed to compositions for oral administration for the treatment of an interferon-responsive disorder which comprises a therapeutically effective amount of at least one alkylated imino sugar having an alkylated side chain and a pharmaceutically acceptable carrier.

### BRIEF DESCRIPTION OF THE FIGURES

FIG. 1 displays the structure of alpha galactosyl ceramide, N-9-oxadecyl-6-methyl-DGJ and N-7-Oxanonyl-6-methyl-DGJ.

FIG. 2 illustrates the fact that alpha galactosyl ceramide and N-9-oxadecyl-6-methyl-DGJ inhibit the secretion of HBV in vitro. A) Alpha galactosyl ceramide inhibits HBV in vitro. B) N-9-oxadecyl-6-methyl-DGJ inhibits the secretion of HBV in vitro.

FIG. 3 illustrates the fact that alpha galactosyl ceramide and N-9-oxadecyl-6-methyl-DGJ, for example, induce components of the host defense pathway in tissue culture.

FIG. 4 illustrates the fact that the amount of HCV RNA is reduced in cells incubated with the combination of interferon and N-methoxynonyl-6-methyl-DGJ, for example.

FIG. 5 shows some of the antiviral effects of interferon.

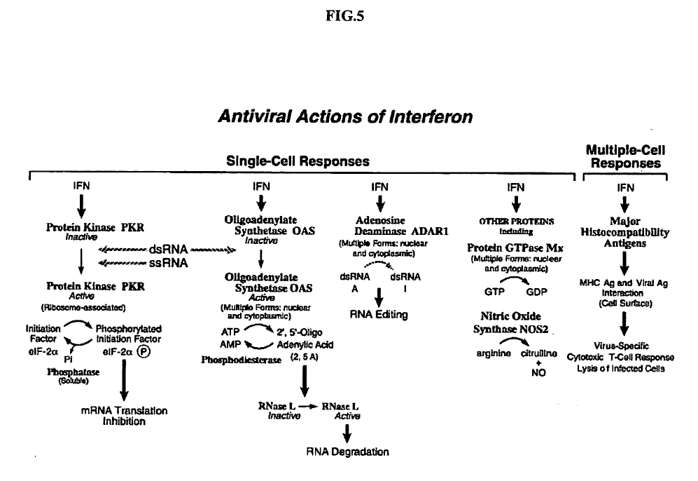

FIG. 6 shows 6-O-nonyl-deoxynojirimycin. The IC50 for 6-O-nonyl-deoxynojirimycin, for example, bovine viral diarrhea virus (BVDV), in tissue cultures of BVDV infected MDBK cells, a surrogate of hepatitis C virus is 3 micro-molar.

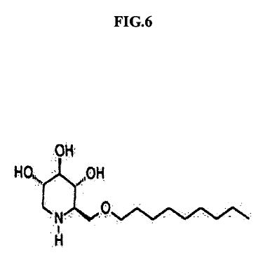

### DETAILED DESCRIPTION OF THE INVENTION

A family of orally available imino sugars with alkyl side chains are described to induce interferon beta and interferon inducible genes and are thus therapeutic for disorders that are responsive to interferon. The following publications are particularly incorporated herein by reference: Mehta, et al., Structure-Activity Relationship of a New Class of Anti-Hepatitis B Virus Agents, Antimicrob Agents Chemother. 2002 December; 46(12): 4004-4008; Lu, et al., The Alkylated Imino Sugar. n-(n-Nonyl)-Deoxygalactonojirimycin Reduces the Amount of Hepatitis B Virus Nucleocapsid in Tissue Culture, J Virol. 2003 November; 77(22): 11933-11940; Mehta, et al., α-Galactosylceramide and Novel Synthetic Glycolipids Directly Induce the Innate Host Defense Pathway and Have Direct Activity against Hepatitis B and C Viruses, Antimicrob Agents Chemother. 2004 June; 48(6): 2085-2090.

The invention described herein relates to small, non protein, molecules, e.g., “alkovirs” that are orally bioavailable and are capable of inducing interferon beta and other biochemical messengers of the interferon pathway that mediate beneficial activity. Since these small molecules described herein are orally available and induce interferons in tissue culture, as a corollary, they have exceptional value in treating interferon responsive disorders when orally administered. Thus, the invention provides a method of treating interferon-responsive disorders, comprising administering to a subject an effective amount of at least one orally-available alkylated imino sugar described herein, for example, containing an alkylated side chain of about 8-10 carbons, wherein the symptoms of the interferon-responsive disorder is controlled.

The invention also provides a method of inducing interferon, or interferon-inducible compounds in a cell, by administering an effective amount of at least one compound described herein.

Unless defined otherwise, all technical and scientific terms used herein have the same meaning as is commonly understood by one of skill in the art to which this invention belongs. All publications and patents referred to herein are incorporated by reference.

The following abbreviations are used herein:

| | |
|---|---|
| DGJ | deoxygalactonojirimycin |
| N9mDGJ | N-9-oxadecyl-6-methyl-DGJ |
| NNDGJ | N-nonyl DGJ |
| N10DGJ | N-decylDGJ |
| N8DGJ | N-octylDGJ |
| DMSO | Dimethylsulfoxide |
| TLR | Toll Like Receptors |
| 2-5-OAS | 2′oxyadenylatesynthestase |
| HBV | hepatitis B virus |
| HbsAg | HBV surface antigen |
| HCV | hepatitis C virus |

Interferons and the biochemical messengers induced by interferon have demonstrated clinical benefit in the treatment of many diseases. Interferons (alpha, beta and gamma) are a family of protein cytokines. Interferons, however, are not bioavailable by means of oral delivery. Moreover, It is not possible to directly therapeutically deliver the biological factors or messengers induced by interferon that mediate its beneficial effects by any pharmaceutical method.

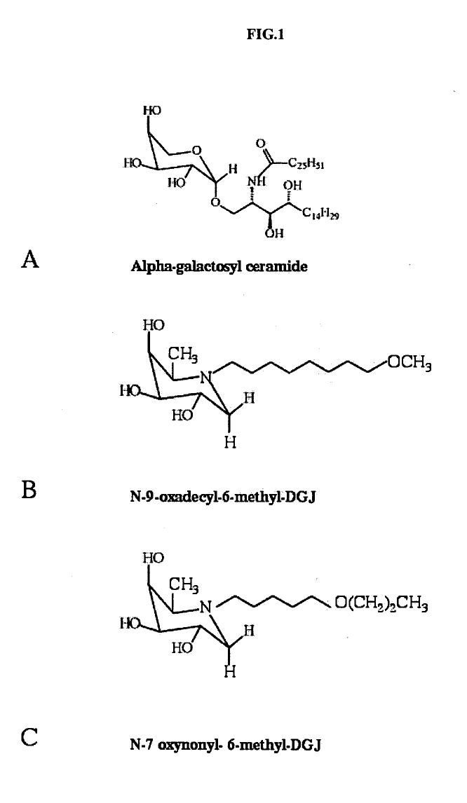

FIG. 1, for example displays the structures of alpha galactosyl ceramide, N-9-oxadecyl-6-methyl-DGJ and N-7-oxanonyl-6-methyl-DGJ. A) Alpha galactosyl ceramide is a naturally occurring glycolipid purified from marine sponges. B-C) The imino sugars used in this study are composed of an imino sugar head group and an alkyl chain. The head groups found in B & C are imino sugar derivatives of galactose (deoxygalactonojirimycins (DGJ)). This is a galactose analogue in which the ring oxygen has been replaced with a nitrogen atom and the anomeric hydroxyl group of galactose is absent. N-7-oxanonyl-6-methyl-DGJ has been shown to have no anti-viral activity against hepatitis B virus. Antimicrob Agents Chemother. 2002 December; 46(12): 4004-4008.

Alpha galactosyl ceramide, shown in FIG. 1A, is a glycolipid derived from marine sponges that is currently in human clinical trials as an anti-cancer agent. It has also been shown to be effective in reducing the amount HBV DNA detected in mice that produce HBV constitutively from a trans-gene. These long chain alkylated sugars bind CD1 molecules on the plasma membranes of diverse cell types and are presented to subset of CD4⁺CD8⁻ or CD4⁻ CD8⁻ T cells that express markers associated with NK cells, and are referred to as NK-T cells. NK-T cells, when activated, secrete cytokines that have anti-viral and anti-tumor properties and thought to mediate an important component of the non MHC dependent immune system.

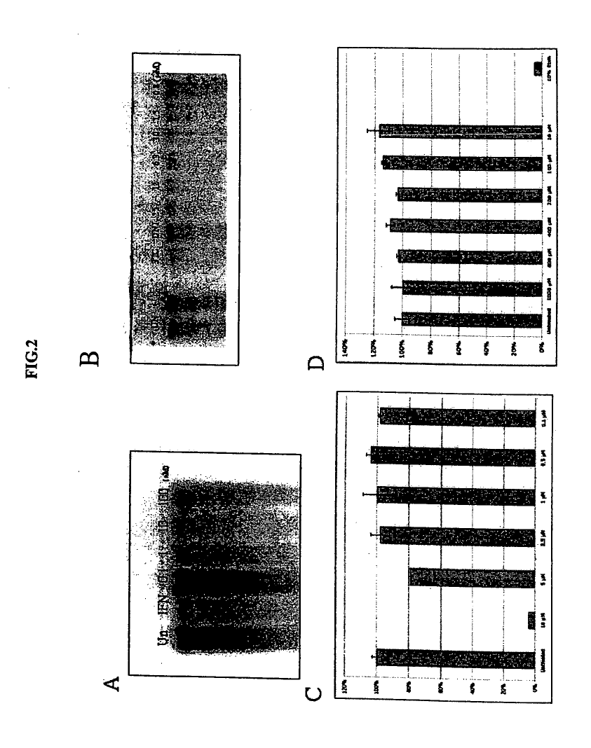

Both alpha galactosyl ceramide and the “alkovirs”, described herein, have anti-hepatitis B virus activity (FIG. 2). As with alpha galactosyl ceramide, our smaller glycolipids do not have any effect upon HBsAg production or secretion, core antigen production or HBV polymerase activity at concentrations that were highly anti-viral FIG. 2B.

Our discovery, reported here, is that the “alkovirs” shown in FIG. 1, for example, have an additional unpredicted activity as a direct anti-viral agent and inducer of the innate host defense pathway, including the induction of interferon beta.

Thus, the invention provides a method of treating interferon-responsive disorders. As used herein, an “interferon-responsive disorder” is any disorder for which symptoms of the disorder are controlled and/or ameliorated upon administering an interferon and/or an interferon-inducible compound to a subject. As used herein, a “subject” is any animal, preferably a mammal, particularly preferably a human being, who is suffering from or who is suspected of having an interferon-responsive disorder.

The innate host defense pathway is activated by means of the rapid induction and high dose desensitization, seen with the glycolipids used here. It was initially believed the glycolipids might work through the TLR (Toll-like receptor) family. However, a key factor of TLR stimulation, NF-KB activation, is not seen with these compounds (data not shown). Thus it is possible, that the glycolipids presented here activate components of the innate host defense pathway by a TLR or NF-KB independent mechanism and analysis of the receptor is currently underway.

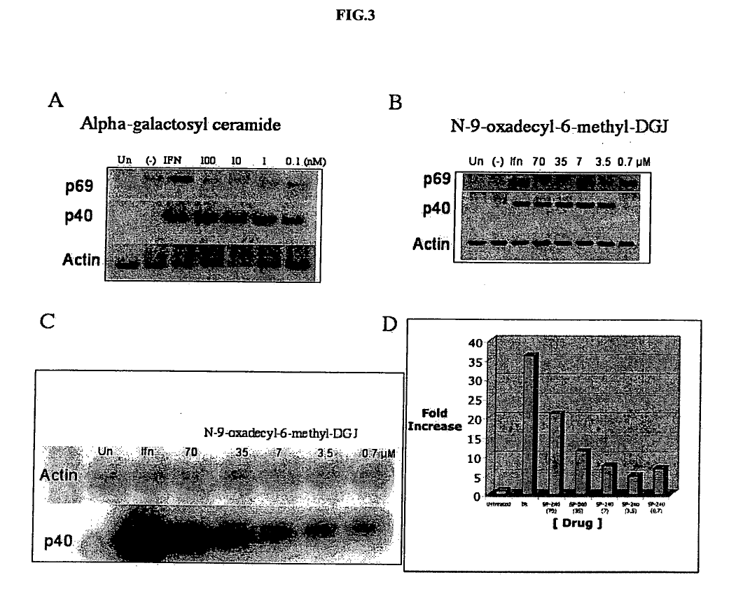

Activation of an innate host defense pathway as shown here is, in some respects, analogous to the phenomenon observed with dsRNA (Guidotti L G, M. A., Mendez H, Koch R, Silverman R H, Williams B R, Chisari F V. 2002. Interferon-regulated pathways that control hepatitis B virus replication in transgenic mice. Journal of Virology 76:2617-2621. In contrast to the situation with dsRNA, however, activation with N-9-oxadecyl-6-methyl-DGJ and alpha galactosyl ceramide appear to induce only a subset of interferon-specific transcripts and is associated with little or no toxicity. FIG. 2-3. In addition, these molecules are orally bio-available, and hence represent orally available therapeutics.

As used herein, interferons include interferon alpha, interferon beta, and interferon gamma. As used herein, “interferon-inducible compounds” include cellular biochemical messengers induced by interferons, such as 2-5-O-A-synthetase and the so called “p40” and p69 human gene products (see Hovnanian A, R. D., Levy E R, Mattei M G, Hovanessian A G. 1999, the entire disclosure of which is herein incorporated by reference, and the human 2′,5′-oligoadenylate synthetase-like gene (OASL) encoding the interferon-induced 56-kDa protein. The OASL maps to chromosome 12q24.2 in the proximity of the 2′,5′-OAS locus; see Genomics 56:362-373, the entire disclosure of which is herein incorporated by reference. Exemplary interferon-responsive disorders include viral infections (e.g., infection with HIV and Hepatitis, including Hepatitis A, B, and C; Herpes, and others); multiple sclerosis and other auto-immune disorders; cancers such as neuroblastomas, multiple myelomas, malignant melanomas, kidney tumors, carcinoid tumors, and ovarian cancer; rheumatoid arthritis and severe respiratory syndrome.

In the practice of the invention described herein an effective amount of one or more orally-available alkylated imino sugar containing an alkylated side chain of at least 7 carbons, for example 8,9, 10, 11, 12, 13, 14, 15, 20, 25 or more carbons, up to about 40, hereinafter called the “inducer”, is administered to a subject. Alkylated side chains which have between about 8 and about 10 carbon atoms in the chain are preferred. The inducer is preferably administered to the patient orally, for example by mouth or intranasally. Alpha galactosyl ceramide and the “alkovirs” shown in FIG. 1 are considered to be “inducers” according to the invention. Other suitable inducers include, for example, deoxygalactonojirimycin (DGJ); N-9-oxadecyl-6-methyl-DGJ (N9mDGJ); N-nonyl DGJ (NNDGJ); N-decyl DGJ (NNDGJ); and N-octylDGJ (N8DGJ). We have also observed that an alkovir, we call: 6-O-nonyl-deoxynojirimycin, which has no glucosidase inhibitory activity but retains antiviral activity, where the alkyl side chain is attached to the carbon penultimate to the nitrogen (FIG. 1D), retains antiviral activity and is likely to act as an inducer of innate host defenses. The present invention reports the importance of an alkyl side chain of 8, 9 or 10 as being a key component of selective (non toxic) biological and antiviral activity.

Compounds which are inducers according to the present invention can be readily identified by one skilled in the art through the assays presented herein.

The present invention particularly encompasses a method of treatment of an interferon-responsive disorder comprising orally administering a composition which comprises a therapeutically effective amount of at least one alkylated imino sugar having an alkylated side chain and a pharmaceutically acceptable carrier wherein symptoms of an interferon-responsive disorder are controlled. Alkylated imino sugars having an alkylated side chain of between 7 and 12 carbon atoms are preferred. Imino sugars, for example, although not limited, may be derivatized, e.g., aza-sugars, selected from the group consisting of galactose, fucose, mannose, glucose, and xylose.

Examples of interferon-responsive disorders within the scope of the present invention include but are not limited to viral infection, multiple sclerosis, auto-immune disease, neuroblastoma, multiple myeloma, malignant melanoma, kidney tumor, carcinoid tumor, ovarian cancer, rheumatoid arthritis, and severe respiratory syndrome.

The inducers according to the invention can be administered to a subject by any suitable means for oral delivery, including solid and liquid dosage forms for administration by mouth, aerosol preparations for intranasal delivery, or suppositories and creams for rectal delivery. Suitable oral dosage forms include liquids, oral solutions or suspensions, immediate release or controlled release tablets, pills, and capsules. One skilled in the art can readily prepare suitable oral dosage forms for administering the present inducers, for example as described in Remingtons's Pharmaceutical Science, 17^{th} Ed., Mack Publishing Co., Easton, Pa., the entire disclosure of which is herein incorporated by reference.

As used herein, an effective amount of the present inducers is an amount sufficient to alleviate or prevent the symptoms of an interferon-responsive disorder. As used herein, to “alleviate the symptoms of an interferon-responsive disorder” means that the clinical or neurologic manifestations of the an interferon-responsive disorder do not worsen over time, and preferably are lessened or eliminated. As used herein, to “prevent the symptoms an interferon-responsive disorder” means that the onset of clinical manifestations of the an interferon-responsive disorder which are expected to occur are delayed or do not occur. The ordinarily skilled physician can readily evaluate the symptoms an interferon-responsive disorder in a subject.

The effective amount of the present inducers can depend on absorption, inactivation and excretion rates of the inducers, as well as other factors known to those of skill in the art. The effective amount can also vary with the penetration of interferon-responsive disorder, and the severity of the symptoms to be alleviated. It is also understood that for any particular subject, specific dosage regimens can be adjusted over time according to individual need. The effective amount of the present inducers can be administered in a single dose, or can be divided into a number of smaller doses to be administered at varying time intervals. Effective amounts of the present inducers to be administered to a subject can be readily determined by one skilled in the art from the examples given below.

A therapeutically effective amount, for a normal sized human, of any of the compounds described herein for single dose oral administration is between about 10 mg and about 500 mg. Dosage is normally between once and three times daily. Single dose oral administration is preferred that is between about 20 mg and about 250 mg. A preferred therapeutically effective amount, however, is generally between about 25 mg and about 1 25mg. N-9-oxadecyl-6-methyl-DGJ or N-7-oxanonyl-6-methyl-DGJ, for example, may effectively be orally administered in amounts between about 20 mg and about 100 mg. 30 mg of either of these compounds, for example, may be administered orally to an adult 3× daily to control an interferon-responsive disorder

The invention thus also provides pharmaceutical compositions comprising one or more inducers of the invention, and a pharmaceutically acceptable carrier. As used herein, “pharmaceutical composition” (also called a “medicament) includes compositions for human and veterinary use. Pharmaceutically acceptable carriers are known in the art, and include any substance which is used in the formulation of a drug dosage form, as are described below. For example, pharmaceutical compositions for oral administration generally comprise an inert or edible carrier, and can be formulated into tablets, troches or capsules (e.g., hard or soft gelatin capsules). Binding agents and/or adjuvant materials can be included as part of the oral pharmaceutical composition. For purposes of the present invention, such binding agents or adjuvant materials are considered pharmaceutically acceptable carriers.

The tablets, pills, capsules, troches and the like of the invention can contain any of the following ingredients, or compounds of a similar nature, all of which are considered pharmaceutically acceptable carriers: a binder such as microcrystalline cellulose, gum tragacanth or gelatin; an excipient such as starch or lactose, a disintegrating agent such as alginic acid, Primogel or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. When the oral dosage form is a capsule, it can contain, in addition to material of the above type, a liquid carrier such as a fatty oil. In addition, oral dosage forms of the invention can contain various other materials which modify the physical form of the dosage unit, for example, coatings of sugar, shellac, or other enteric agents.

Liquid oral dosage forms of the invention can comprise an elixir, solution, suspension, syrup, wafer, chewing gum or the like. A syrup can further comprise a sweetening agent (such as sucrose or other sugar, or artificial sweetener such as aspartame or xylitol or Splenda®) and certain preservatives, dyes and colorings and flavors, all of which are considered pharmaceutically acceptable carriers or excipients.

Gelatin capsules can contain the present inducers and suitable pharmaceutically acceptable carriers, such as lactose, sucrose, mannitol, starch, cellulose derivatives, magnesium stearate and steric acid. Similar pharmaceutically acceptable carriers can be used to make compressed tablets. Both tablets and capsules can be manufactured for the sustained release of the present inducers over a period of time; e.g., minutes to hours. Compressed tablets can be sugar coated or film coated to mask any unpleasant taste and protect the tablet from the atmosphere, or they can be enteric coated for selective disintegration in the gastrointestinal tract.

Suppositories can contain the present inducers in an oleaginous or water-soluble base. Suitable oleaginous bases include cocoa butter and other fats with similar properties, Suitable water-soluble bases include the polyethylene glycols.

The invention also provides a method of inducing interferon, or interferon-inducible compounds, in a cell by administering to that cell an effective amount of at least one inducer of the invention. The cell can be in vitro or in vivo, or can be treated ex vivo and reimplanted into a subject. The cell can be any cell which produces interferons or interferon-inducible compounds, in particular cells of the immune system such as leukocytes or dendritic cells.

As used herein, an effective amount of an inducer of the invention which induces interferon or interferon-inducible compounds in a cell is any amount which causes a detectable amount of interferon or interferon-inducible compounds to be produced in that cell. One skilled in the art can readily determine an effective amount, for example by assaying the cells for production of interferon or interferon-inducible compounds by any suitable technique. Suitable techniques for detecting production of interferon or interferon-inducible compounds include techniques for detecting the amount of RNA produced in a cell, such as R/T PCR or Northern blot, or techniques for detecting the amount of protein produced in a cell, such as immuno-bases assays (e.g., ELISA, ELISPOT, Western blot). Performing these techniques is within the skill in the art. Also, suitable effective amounts can be determined by one skilled in the art from the examples presented below.

We have shown that small orally available glycolipid mimetics, such as N-methoxynonyl-6-methyl-DGJ, can directly activate cellular defense genes (such the small 2′,5′ OAS) and reduce the amount of HBV and HCV replication, without recruitment of any cells other then those infected. Since the synthetic glycolipids that stimulate this response could be mimetic for pathogen glycolipids, we propose that hepatocytes have the ability themselves to autogenously recognize and react defensively to foreign pathogen molecules without assistance from any other immunological cells and perhaps this represents a very primitive arm of the host defense system. Thus these synthetic glycolipids represent a new class of orally available small molecules that may have therapeutic value in all cases where interferon induction is useful.

Compositions and methods of the present invention are preferred wherein the inducer comprises a side chain attached to any reactive atom in its sugar ring. Inducers are preferred which comprises any derivation of an imino sugar head group and an alkyl side chain longer than 8 carbon atoms. Preferred inducers are, for example, N-nonyl-DGJ, N8-DGJ, and N10-DGJ. Methods, compositions and articles of manufacture of the present invention, e.g., corresponding to treatment of an interferon-responsive disorder comprising orally administering a composition which comprises a therapeutically effective amount of at least one alkylated sugar having an alkylated side chain, moreover encompass methods where the inducer is alpha gal ceramide or other glycolipid mimetics. Methods of the invention described herein are particularly drawn toward wherein the interferon-responsive disorder is responsive to interferon alpha. Methods of the invention described herein are particularly drawn toward wherein the interferon-responsive disorder is responsive to interferon beta. Methods of the invention described herein are particularly drawn toward wherein the interferon-responsive disorder is responsive to interferon gamma. Methods of the invention described herein are drawn toward wherein the interferon-responsive disorder is selected from the group consisting of viral infection, multiple sclerosis; neuroblastomas, multiple myelomas, malignant melanomas, kidney tumors, carcinoid tumors, ovarian cancer; rheumatoid arthritis and severe respiratory syndrome. Methods of the invention described herein are particularly drawn toward wherein the interferon-responsive disorder is multiple sclerosis. Methods of the invention described herein are particularly drawn toward wherein the interferon-responsive disorder is multiple myeloma. Methods of the invention described herein are particularly drawn toward wherein the interferon-responsive disorder is malignant melanoma. Methods of the invention described herein are particularly drawn toward wherein the interferon-responsive disorder is ovarian cancer. Methods of the invention described herein are particularly drawn toward wherein the interferon-responsive disorder is a kidney tumor. Methods of the invention described herein are particularly drawn toward wherein the interferon-responsive disorder is a carcinoid tumor. Methods of the invention described herein are particularly drawn toward wherein the interferon-responsive disorder is hepatitis B or hepatitis C infection. Methods of the invention described herein are particularly drawn toward wherein the interferon-responsive disorder is rheumatoid arthritis. Methods of the invention described herein are particularly drawn toward wherein the interferon-responsive disorder is neuroblastoma.

Methods, compositions and articles of manufacture of the present invention, e.g., corresponding to treatment of an interferon-responsive disorder comprise administering a composition which comprises a therapeutically effective amount of at least one alkylated sugar described herein, having an alkylated side chain, described herein, to a cell, particularly wherein the cell is an immune cell or a dendritic cell, wherein the cell is in vitro or in vivo.

The direct anti-viral activity of alpha galactosyl ceramide and orally available glycolipids described herein were initially tested in tissue culture using the stable, HBV producing cell line, Hep G2 2.2.15 cells (Sells, M. A., Chen, M. L., Acs, G. 1987. Hep G2 cells transfected with cloned hepatitis B virus DNA. Proc. Natl. Acad. Sci. USA 84:1005-1009, the entire disclosure of which is herein incorporated by reference in its entirety). The results, shown in FIG. 2A, clearly demonstrate that alpha galactosyl ceramide and the smaller orally available glycolipid N-9-oxadecyl-6-methyl-DGJ effectively reduce the amount of HBV detected in the culture medium in a dose dependent manner.

FIG. 2 illustrates the fact that alpha galactosyl ceramide and N-9-oxadecyl-6-methyl-DGJ inhibit the secretion of HBV in vitro. A) Alpha galactosyl ceramide inhibits HBV in vitro. B) N-9-oxadecyl-6-methyl-DGJ inhibits the secretion of HBV in vitro. Briefly, Hep G2 2.2.15 cells, which secrete hepatitis B virus, were either left untreated or treated with 10³ iU/mL of interferon alpha (2a/2b) or 0.1 nM to 100 nM of alpha galactosyl ceramide for three days and the amount of HBV specific DNA detected in the culture medium using a method which discriminates between enveloped and non enveloped viral particles. The HBV rc DNA, which decreases with both interferon and alpha galactose ceramide treatment is indicated. B) N-9-oxadecyl-6-methyl-DGJ inhibits the secretion of HBV in vitro. Hep G2 2.2.15 cells were left untreated or treated with varying doses of the synthetic glycolipid N-9-oxadecyl-6-methyl-DGJ (0.8 μM to 25 μM) for 3 days and the level of HBV secreted into the culture medium detected as before. 3TC lamivudine is a nucleoside analogue and inhibits the secretion of HBV and is used as a control. The HBV rc DNA, which decreases with N-9-oxadecyl-6-methyl-DGJ, interferon, and alpha galactosyl ceramide treatment is indicated. C-D) Cells treated with alpha-galactosyl ceramide or the synthetic glycolipid N-9-oxadecyl-6-methyl-DGJ were tested for viability using the standard MTT toxicity test. For panel C: X-axis: From left to right: Untreated cells; Alpha galactosyl ceramide at 10 μM, 5 μM, 2.5 μM, 1 μM, 0.5 μM, 0.1 μM. Y-axis, percent viability as compared to untreated group. For Panel D: X-axis: From left to right: Untreated cells; N-9-oxadecyl-6-methyl-DGJ at 2000 μM, 800 μM. 400 μM, 200 μM, 100 μM, 10 μm. Y-axis, percent viability as compared to untreated group.

FIG. 3 illustrates the fact that alpha galactosyl ceramide and N-9-oxadecyl-6-methyl-DGJ, for example, induce components of the host defense pathway in tissue culture. Hep G2 cells were treated with the indicated doses of A) alpha galactosyl ceramide or with B) N-9-oxadecyl-6-methyl-DGJ (b) for 16 hours and the induction of the medium (p69) and small (p40) isoforms of the 2′,54 OAS gene determined by R/T PCR. For each set, actin levels were also monitored as a control for loading. The negative control in both panels is the imino sugar N-7-oxanonyl-6-methyl-DGJ, which has no anti-viral activity(14) and as this figure shows, does not induce components of the host defense pathway. C) Southern blot- R/T PCR of RNA from Hep G2 cells treated with the indicated concentration of N-9-oxadecyl-6-methyl-DGJ or with 10³ iU/mL interferon alpha (2a/2b) for 16 hours. Briefly, limited PCR was performed as described for 5, 10, 15 cycles and the PCR products transferred to nylon membrane before hybridization with a 1377 bp cDNA probe from nucleotides 1 to 1377 from the published OAS-40/46 gene (accession #X02874). Actin controls were performed in the same manner. The results of the 10 cycle PCR and hybridization are shown. D) Quantification of the blot as shown in FIG. 3C and indicates that N-9-oxadecyl-6-methyl-DGJ induced a 20 fold induction of the 2′,5′ OAS gene at 70 μM with lower concentrations giving a dose dependent 2-15 fold increase in 2′,5′ OAS gene expression.

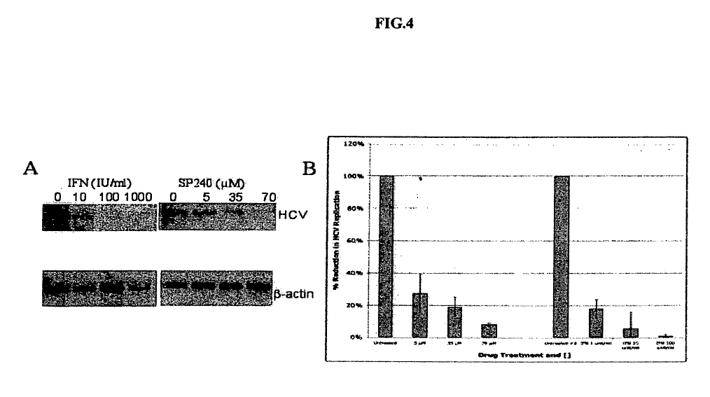

FIG. 4 illustrates the fact that the amount of HCV RNA is reduced in cells incubated with the combination of interferon and N-methoxynonyl-6-methyl-DGJ, for example. 9-13 cells, which harbor the 8 kb HCV bi-cistronic RNA replicon were incubated for 48 hours in the absence or presence of human interferon alpha or N-9-oxadecyl-6-methyl-DGJ (SP-240) at the indicated concentration. A) Total RNA was isolated, resolved through agarose gels and hybridized to a radioactive probe specific for HCV NS5B. After washing, the blot was and re-probed with radioactive beta-actin specific sequences to control for loading of lanes. B) Quantification of reduction of three separate experiments using N-9-oxadecyl-6-methyl-DGJ (Sp-240). The IC50 for N-9-oxadecyl-6-methyl-DGJ is 1.5 μM.

### EXAMPLES

### Example I

### Alpha Gal Ceramide and the Synthetic Glycolipid (“alkovir”), N-9-oxadecyl-6-methyl-DGJ Exhibit Anti-Viral Activity at Varying Doses

FIG. 2A shows that the addition of alpha galactosyl ceramide at doses of 1 μM to 100 nM to tissue culture cells inhibited the secretion of enveloped HBV. In this assay, alpha interferon is used as a control and also inhibits secretion effectively at a dose of 10³ iU/m. It is noted that the potency of alpha galactosyl ceramide was dependent upon the formulation of its delivery; dissolution in the lipophilic solvent provided by the supplier (intended to promote intracellular delivery) actually reduced potency (data not shown) suggesting that a surface receptor is involved. Similar to alpha galactosyl ceramide, and consistent with our previous reports regarding this compound class the synthetic glycolipid, N-9-oxadecyl-6-methyl-DGJ also exerted anti-viral activity at varying doses (FIG. 2B) (Mehta, A., Conyers, B., Tyrrell, D. L. J., Walters, K. A., Graham A. Tipples, G. A., Dwek, R. A., Block, T. M. 2002. Structure-activity relationship of a new class of anti-hepatitis B virus agents. Antimicrobial Agents and Chemotherapy 46:4004-4008).

Method:

Hep G2 2.2.15 cells were kindly provided by Dr. George Acs [Mt. Sinai Medical College, NY, N.Y.] and maintained in the same way as Hep G2 cells. The HCV subgenomic replicon cell line 9-13, a kind gift of Dr. Bartenschlager (12), was cultured in Dulbecco's Modified Eagle Media (DMEM) (Invitrogen Corporation, Carlsbad, Calif., USA) containing 10% fetal calf serum, 1% penicillin-streptomycin, 1% non-essential amino acids and 0.5 mg/ml Geneticin. Cells were maintained at sub-confluency prior to splitting. All compounds were dissolved in sterile double distilled water unless noted.

Analysis of DNA secreted from tissue culture cells was performed by a method which would discriminate between enveloped and unenveloped virions. Briefly, Hep G2 2.2.15 cells were seeded at 85-90% confluence in T-25 flasks and 5 days later the indicated drug was added at the indicated concentrations. After 3 days virus was concentrated from supernatant with poly-ethylene glycol (PEG). Virus was resuspended in 200 μl of 10 mM TRIS [pH 7.9], 10 mM EDTA [pH 8.0], and 10 mM MgCl₂. Proteinase K was added to a final concentration of 750 μg/ml and the samples incubated for 1 hour at 37° C. After 1 hour, SQ1 DNase (Promega, Madison, Wis., USA) was added to each tube to a final concentration of 50 units/ml and incubated at 37° C. for 1 hour. SDS was added to a final concentration of 1% and more Proteinase K added to a final concentration of 500 μg/ml and the reaction allowed to proceed at 37° C. for 4 hours. DNA was purified by phenol/chloroform extraction followed by isopropanol precipitation. DNA was separated by electrophoreses on a 1.0% agarose gel, transferred to a nylon membrane and probed with ³²P labeled HBV probes as described elsewhere. HBV specific bands were subsequently identified and quantified by phosphor image analysis (Bio-Rad, Hercules, Calif., USA).

### Example II

### The Alpha Galactosyl Ceramide and the Synthetic Glycolipid N-9-oxadecyl-6-methyl-DGJ Did Not Exhibit Cytotoxicity at Concentrations and Conditions Under Which They Demonstrated Antiviral and Interferon Induction Activity, Demonstrating Useful Selectivity

The cytotoxicity profiles of alpha galactosyl ceramide and the synthetic glycolipid N-9-oxadecyl-6-methyl-DGJ were examined in parallel (and under the same conditions), as were the anti-viral profiles, and the results shown in FIGS. 2C-D. In these experiments, the CC50 of alpha galactosyl ceramide is 8 μM and >2000 μM for N-9-oxadecyl-6-methyl-DGJ. Since the IC50 values for alpha galactosyl ceramide and N-9-oxadecyl-6-methyl-DGJ were approximately 0.4 nM and 1 μM respectively, it is safe to say that the anti-viral activity of these compounds occurs at concentrations well below the amount at which toxicity was observed, thus demonstrating selectivity for viral functions.

Method to Determine Toxicity of Compounds:

Toxicity was determined by mitochondrial toxicity testing (MTT) of cells that had been exposed to compounds in a manner similar to the anti-viral assays except cells were treated in 24 well trays. Briefly, Hep G2 2.2.15 cells were treated with drug as indicated in the figure legends for 3 days, the media removed and replaced with 100 μl of a 10 mg/ml solution of tetrazolium bromide [[3-[4,5-dimethylthiazol-2-yl]-2,5-diphenyltetrazolium bromide, Sigma Chemicals] for one hour at 37° C. Addition of 100-200 μl of dimethylsulfoxide (DMSO) led to color development. Supernatants were removed and placed in a 96 well tray for analysis. OD values were read at 590 nm.

### Example III

### Neither Alpha Galactosyl Ceramide or N-9-oxadecyl-6-methyl-DGJ had any Detectable Effect Upon HBsAg Production or Secretion, Core Antigen Production or Secretion or HBV Polymerase Activity at Concentrations that were Highly Anti-Viral

This indicates both that the compounds were both well tolerated at these concentrations and that the anti-viral activity cannot be explained by a direct effect upon the synthesis of viral products but rather through the activation of a cellular defense mechanism.

### Example IV

### Induction of the Innate Host Defense Pathway by Alpha Galactosyl Ceramide and N-9-oxadecyl-6-methyl-DGJ

The ability of alpha galactosyl ceramide and N-9-oxadecyl-6-methyl-DGJ to directly induce the innate host defense pathway in tissue culture was determined by analysis of the induction of the 2′-5′ oligoadenylate synthetase genes (2′,5′ OAS) using an RT PCR based methodology. As FIG. 3A shows, interferon alpha is a potent inducer of both the medium (p69) and small (p40) 2′,5′ OAS genes. In contrast, alpha galactosyl ceramide and the synthetic glycolipid, N-9-oxadecyl-6-methyl-DGJ induced only the small (p40) 2′,5′ OAS gene expression over a wide dose range. Further examination and quantification using a southern blot-R/T PCR based methodology has indicated that N-9-oxadecyl-6-methyl-DGJ induced a 20 fold induction of the 2′,5′ OAS gene at 70 μM with lower concentrations giving a dose dependent 2-15 fold increase in 2′,5′ OAS gene expression (FIG. 3, C-D). In contrast, a compound that is structurally similar to N-9-oxadecyl-6-methyl-DGJ, but has limited anti viral activity against HBV (N-7-oxanonyl-6-methyl-DGJ)(14), did not detectably induce 2′,5′ OAS gene expression (FIG. 3, A-B). This result provides evidence for the chemical specificity of this activation, as these compounds only differ in one carbon and the localization of the oxygen in the alkyl tail (compare FIG. 1, B&C). It is noted that the anti-viral activity seen with this compound class correlates with the induction of the small 2′,5′ OAS gene. That is, compounds and doses that are anti-viral induce the small 2′,5′ OAS gene, while compounds and doses with no anti-viral activity do not (FIGS. 2 & 3 and data not shown).

Method for Analysis of 2′,5′ OAS Genes

Briefly, Hep G2 cells were incubated with either alpha galactose ceramide or the synthetic glycolipids shown in FIG. 1 for the desired length of time and the total RNA harvested using Tri-reagent as per manufactures directions (Gibco-BRL, Rockville, Md., USA). RNA samples were further purified using the Ambion DNA™ free kit, (Ambion, Inc., Austin, Tex., USA) before reverse transcriptase (RT) PCR with the PCR conditions and primers exactly as reported in the literature. PCR was performed in the absence of (RT) for 50 cycles to ensure no DNA contamination. Dilution experiments were used to ensure that PCR was within the linear range of the assay. Southern blot-R/T PCR of RNA from Hep G2 cells treated with the indicated concentration of N-9-oxadecyl-6-methyl-DGJ or with interferon alpha (2a/2b) for 16 hours was also performed to allow for quantification of induction. Briefly, limited PCR was performed as described above for 5, 10, 15 cycles and the PCR products transferred to nylon membrane. Hybridization was carried out using a 1377 bp cDNA probe from nucleotides I to 1377 from the published OAS40/46 gene (accession #X02874). OAS-40/46 specific bands were identified and quantified by phosphor image analysis [Bio-Rad, Hercules, Calif.].

### Example V

N-9-oxadecyl-6-methyl-DGJ, for example, inhibits the hepatitis C virus (HCV) replicon, which is highly sensitive to interferons.

As N-9-oxadecyl-6-methyl-DGJ could induce the innate host defense pathway, it was of interest to determine its anti-viral effect against other viruses, such as hepatitis C virus (HCV). Clone 9-13 is a Huh7 derived cell line that constitutively expresses the bicistronic HCV subgenomic replicon(12). Replication of the HCV sub-genome is dependent upon the viral replicase but not envelope proteins (nor the HCV p7 protein) since the structural genes were deleted and as shown in FIG. 4, is sensitive to interferon alpha. Since replication of HCV RNA in 9-13 cell is sensitive to interferon alpha, and N-9-oxadecyl-6-methyl-DGJ induces an arm of the interferon pathway, it was hypothesized that N-9-oxadecyl-6-methyl-DGJ would have an anti-viral effect upon HCV in this cell line. This possibility was tested by examining the amount of HCV RNA in 9-13 cells as a function of incubation in varying concentrations of our lead compound, N-9-oxadecyl-6-methyl-DGJ. The results, shown in FIG. 4, demonstrate a clear dose dependent reduction in the steady state level of HCV RNA, with an IC50 for N-9-oxadecyl-6-methyl-DGJ of 1.5 μM. Beta-actin protein and RNA were used as controls in these experiments. Thus, as predicted, N-9-oxadecyl-6-methyl-DGJ is inhibitory for HCV replicons.

Method for Inhibiting HCV and Detection of HCV RNA.

9-13 cells were seeded in T25 flask at 3×10⁶ cells. After allowing for adhesion of the cells, the indicated concentration of IFN or N-9-oxadecyl-6-methyl-DGJ was added and the cells were incubated for 48 hours and the RNA isolated using the RNAeasy™ kit (Qiagen, Valencia Calif., USA). Northern blot analysis was done to analyze HCV replicon RNA level.

Briefly, 2 μg total RNA was electrophoresed through a 1.0% agarose gel containing 2.2 M formaldehyde, transferred to a nylon membrane and immobilized by Lw cross-linking (Stratagene). Hybridization was carried out using an alpha-[³²P]CTP-labeled probe with random primers on a 2 kb NS5B DNA fragment in a quick hybridization solution (Amersham Bioscience, Piscataway, N.J.) for 16 h at 65° C. The membranes were washed once in 2×SSC/0.1% SDS for 30 min at room temperature and twice in 0.1×SSC/0.1% SDS for 30 min at 65° C. Radioactive signal was identified and quantified by phosphor image analysis (Bio-Rad, Hercules, Calif.).

Method of Western Blot Analysis:

Western blot analysis was done as is known in the art. A monoclonal antibody to NS5A, a kind gift of Dr. C. Liu (Univ. Florida, Gainsville, Fla., USA), was used to measure the viral protein level.

All publications and patents referred to herein are incorporated by reference. Various modifications and variations of the described subject matter will be apparent to those skilled in the art without departing from the scope and spirit of the invention. Although the invention has been described in connection with specific embodiments, it should be understood that the invention as claimed should not be unduly limited to these embodiments. Indeed, various modifications for carrying out the invention are obvious to those skilled in the art and are intended to be within the scope of the following claims.

1. A method of treatment of a pathophysiological condition by induction of an innate host defense pathway, comprising orally administering a composition which comprises a therapeutically effective amount of at least one alkylated imino sugar having an alkylated side chain, and a pharmaceutically acceptable carrier, wherein symptoms of the condition are controlled.

2. A method of treatment according to claim 1 wherein the pathophysiological condition is an interferon-responsive disorder and the alkylated imino sugar has an alkylated side chain of between 7 and 12 carbon atoms.

3. A method of treatment according to claim 2 wherein the interferon-responsive disorder is selected from the group consisting of viral infection, multiple sclerosis, auto-immune disease, neuroblastoma, multiple myeloma, malignant melanoma, kidney tumor, carcinoid tumor, ovarian cancer, rheumatoid arthritis, and severe respiratory syndrome.

4. A method of treatment according to claim 3 wherein the interferon-responsive disorder is multiple sclerosis.

5. A method of treatment according to claim 1 wherein the imino sugar is a derivative selected from the group consisting of aza-galactose, aza-fucose, aza-mannose, aza-glucose, aza-xylose, and aza-mannose.

6. A method of treatment according to claim 5 wherein the imino sugar is a derivative of galactose.

7. A method of treatment according to claim 6 wherein the alkylated imino sugar is a deoxygalactonojirimycin (DGJ).

8. A method of treatment according to claim 7 wherein the alkylated imino sugar is selected from the group consisting of N-9-oxadecyl-6-methyl-DGJ and N-7-oxanonyl-6-methyl-DGJ.

9. A method of treatment according to claim 2 wherein the alkylated side chain is of between 8 and 10 carbon atoms.

10. A method of treatment according to claim 9 wherein the alkylated side chain is 8 carbon atoms.

11. A method of treatment according to claim 9 wherein the alkylated side chain is 9 carbon atoms.

12. A method of treatment according to claim 9 wherein the alkylated side chain is 10 carbon atoms.

13. A method of treatment according to claim 1 wherein the alkylated imino sugar is selected from the group consisting of 6-O-septyl-deoxynojirimycin, 6-0-octyl-deoxynojirimycin, 6-O-nonyl-deoxynojirimycin, N-9-oxadecyl-6-methyl-DGJ (N9mDGJ), N-nonyl DGJ (NNDGJ), N-decylDGJ (N10DGJ), and N-octylDGJ (N8DGJ).

14. A method of treatment according to claim 1 wherein the alkylated imino sugar is oxygenated.

15. A method of treatment according to claim 14 wherein the alkylated imino sugar is selected from the group consisting of 6-O-septyl-methoxy-deoxynojirimycin, 6-O-octyl-methoxy-deoxynojirimycin, 6-O-nonyl-methoxy-deoxynojirimycin, and 6-O-decyl-methoxy-deoxynojirimycin, or is a 6-O-nonyl-deoxynojirimycin compound, or any compound with an imino sugar head group and side chain attached to the carbon penultimate to the nitrogen, or any organic compound with a side chain of 7-9 carbons.

16. A method of treatment according to claim 1 wherein the therapeutically effective amount is between about 10 mg and about 500 mg.

17. A method of treatment according to claim 2 wherein the therapeutically effective amount is between about 20 mg and about 250 mg.

18. A method of treatment according to claim 8 wherein the therapeutically effective amount is between about 25 mg and about 125 mg.

19. A container comprising a composition which comprises a therapeutically effective amount of at least one alkylated imino sugar having an alkylated side chain and a pharmaceutically acceptable carrier; and, instructions for oral administration of the composition for the treatment of an interferon-responsive disorder.

20. A composition for oral administration for the treatment of an interferon-responsive disorder which comprises a therapeutically effective amount of at least one alkylated imino sugar having an alkylated side chain and a pharmaceutically acceptable carrier.
